(19) United States
(12) Patent Application Publication
(10) Pub. No.: US 2006/0189938 A1
(43) Pub. Date: Aug. 24, 2006

(54) Automatic injector with a release lock

(76) Inventors: Edgar Hommann, Grossaffoltern (CH); Pascal Baer, Detligen (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/355,910

(22) Filed: Feb. 16, 2006

(30) Foreign Application Priority Data
Feb. 18, 2005 (DE) 10 2005 007 614.9

(51) Int. Cl. A61M 5/20

(52) U.S. Cl. 604/137

(57) ABSTRACT

An injection device including a contact element which is pressed onto a body tissue for an injection, a release for initiating product dispensing, which contact element and release are respectively displaceable along a longitudinal axis of the device between an initial position and an end position, wherein a movement of the release in at least one of the initial position of the contact element and the end position of the release can be prevented.

### CROSS-REFERENCE TO RELATED APPLICATION(S)

This application claims the benefit of German Application No. DE 10 2005 007 614.9, filed on Feb. 18, 2005, the contents of which are incorporated herein in its entirety by reference.

### BACKGROUND

The invention relates to a devices for administering, dispensing, delivering or injecting a substance and to methods of making and using such devices. More particularly, it relates to an injection device for injecting an injectable product. More particularly, the injection device may be an automatic injector.

With respect to devices for administering injectable products, there is a desire for the most compact construction possible, in particular for a shortest possible length as measured in the forward drive direction of the injection mechanism, which frequently includes a plunger or piston rod, in order to improve handling. This desire is more pressing in the case of automatic injectors specifically, because automatic injectors are generally already longer than conventional injection devices, incorporating simple tips because the piercing cannula has to be moved relative to a housing of the respective device in the forward drive direction by an additional amount corresponding to the desired piercing depth for the injection. Particularly in the case of disposable injection devices, a mechanism is needed which is relatively simple but which offers an appropriate degree of safety. Such devices should also have means to indicate that it has been used or is suitable for a single administration due the nature of its design.

### SUMMARY

Accordingly, one objective of the invention is to provide an injection device with a relatively simple and safe mechanism which provides an indication that the device has been used.

In one embodiment, the present invention comprises an injection device comprising a contact element which is pressed onto a body tissue for an injection, a release for initiating product dispensing, which contact element and release are respectively displaceable along a longitudinal axis of the device between an initial position and an end position, wherein a movement of the release in at least one of the initial position of the contact element and the end position of the release can be prevented or blocked.

In one embodiment, the invention relates to an injection device, in particular an automatic injector, for dispensing a product from a container which is accommodated or can be accommodated by the injection device. An automatic injector is a device which does not require a user to effect the dispensing movement himself, i.e. the forward drive movement of a plunger housed in the device. He merely has to operate a mechanism, which then automatically drives the plunger in the forward drive direction of the device. The injection device has a contact element, which is pushed onto a body tissue for an injection. Such a contact element may be a needle protector sleeve or a needle cover element, for example. In principle, the contact element may also be a structure projecting out in the distal direction of the injection device. By “distal” direction is meant the end of the device pointing in the direction opposite the piercing direction of the needle, referred to as the “proximal direction”. The container accommodating a product may be an ampoule, for example, to which a needle can be attached and which accommodates a plunger which is displaceable along a longitudinal axis of the ampoule, for example, which is disposed at the proximal end of the ampoule and closes the ampoule off at this end. The injection device has a release means which, when operated accordingly, is able to initiate dispensing of a product from the ampoule. The contact element and the release means are each displaceable along a longitudinal axis of the injection device, in particular relative to a housing, between an initial position and an end position. The contact element is preferably disposed at the distal end and the release means at the proximal end of the device and are mounted so as to be longitudinally displaceable in the housing. The housing may be made in one piece, for example, but in one preferred embodiment is made of two parts comprising a proximal and a distal housing part. The two housing parts may be releasably or non-releasably connected to one another by means of a housing lock.

When the contact element is in the initial position, the contact element projects out in the proximal direction of the injection device and is displaceable in the distal direction. When the contact element is in the end position, the contact element does not project out from the distal end of the device or does so to a much lesser degree than in the initial position and is essentially no longer displaceable in the distal direction. When the release element is in the initial position, the release element projects proximally out of the housing and can then be moved distally into the housing. When the release means is in the end position, the release means is moved proximally into the housing compared with the initial position, in which case a release mechanism will have been operated so that product has been or can be dispensed.

As proposed by the invention, a movement of the release means can be blocked (or prevented or locked) in at least one of the initial position of the contact means or the end position of the release means. Consequently, before operating the release means, the contact element must be moved in the proximal direction of the device, in particular pressed against a body tissue, in order to operate the release means and thus initiate dispensing of the product.

By preference, a lock member is able to block the movement of the release means along the longitudinal axis when the contact element is in the initial position. The lock member may preferably be displaceable into and out of the locking engagement transversely to the longitudinal axis of the device. The lock member may be a resilient component, for example, such as a resilient arm. The arm preferably extends more or less in the longitudinal direction or at a slight angle to the longitudinal direction so that a force applied to the release means can be absorbed by the arm in such a way that the normal force generated in the arm more or less corresponds to the force applied to the release element. The lock member respectively arm may be forced apart supported or braced in the direction opposite that in which the release means is operated, for example. In some preferred embodiments, the lock member and the release means each form part of a cam mechanism, in which case a movement of the release means in the direction of the longitudinal axis can cause a movement of the lock member directed transversely to the longitudinal axis. In an arrangement where the lock member is disposed on an arm, for example, the lock member can be moved out of the locking engagement with the release means against the spring force of the arm. An advantage is that the lock member and the release means each have a surface, in which case the surface of the one is able to slide on the surface of the other due to the movement of the release means.

In a preferred embodiment, the device has a release member, which is displaceable in the direction of the longitudinal axis of the device, for example. The release member can be coupled with the contact element, for example, or may be formed by the contact element, so that a movement of the contact element is specifically able to cause a movement of the release member relative to the lock member. In order to make the device easier to assemble, the release member may be disposed on a longitudinally displaceable sliding sleeve in the proximal housing part, for example, and the contact element in the distal housing part, for example. It may be preferable if the contact element and the release means can be reset to their initial positions in opposite directions, especially if only one of the two has not been operated or has not been fully operated or moved into its end position. An energy storing means may be used for this purpose, for example a return spring, which may be disposed between the contact element or a sliding sleeve coupled with the contact element and the release means.

In a preferred embodiment, when the lock member is in a position in which it is blocking movement of the release means, the release member holds the lock member in a locked engagement with the release means. The release member preferably holds the lock member in a locked engagement if the contact element is at least in an initial position, or when the contact element is in any position except the end position. In particular, the release member is able to prevent the lock member from moving out of engagement with the release means. Since the lock member is able to move out of the locked engagement transversely to the longitudinal axis of the device for example, it is of advantage to dispose the release means on a level with the lock member in the longitudinal direction if the release means is intended to be locked. The release means should be locked when the contact element is not fully in its end position, for example.

In one advantageous embodiment, the release means is displaceable along the longitudinal axis relative to the housing and the lock member is coupled with the housing so that the lock member can be moved out of the locked engagement with the release means away from the release means against the elastic force. The cam mechanism preferably may be designed so that a longitudinal displacement of the release means does not cause any self-locking due to friction between the mechanism surfaces of the release means and lock member.

The injection device may preferably a latch member able to block movement of the release means when the lock member is in a position in which it is not blocking the movement of the release means. In another preferred embodiment, the latch member is able to block the release means in its end position or in a position in which it has released product dispensing. For example, the release means can engage in a groove, a recess or similar, for locking purposes, for example. The latch member is advantageously resilient or displaceable against an elastic force. The latch member may be provided on a resiliently mounted arm, for example. Preferably, a user of the device is not able to access the latch member directly from outside, which means that once the latch member is latched in the release means, it can no longer be released from its engagement with the release means other than by breaking the injection device.

The injection device may also have an unlatching member, which is retained in a retaining engagement with a plunger rod by the release means in order to block a dispensing movement of the plunger rod. Particularly in the case of automatic injectors, the plunger rod is in a biased position due to a drive mechanism prior to product dispensing. The drive mechanism may be a drive spring, for example. To establish the retaining engagement, the plunger rod may have a retaining shoulder, for example, which may be preferably disposed at the proximal end of the plunger rod. The release element may partially surround the plunger rod or the region of the retaining shoulder of the plunger rod, for example. The unlatching element may form a stop for the retaining shoulder acting in the longitudinal direction of the device. The radial distance between the release means and retaining shoulder may be shorter than the dimension of the unlatching member pointing in this direction. Since the unlatching member is disposed so that it essentially can not be displaced axially relative to the housing, the unlatching member is able to block the dispensing movement of the plunger rod when the release means is in the initial position.

The biased drive spring, which may be fixedly supported to the housing at one end and supported on the plunger rod at the other end, is not able to move the plunger rod in the dispensing direction when in the blocked state. The plunger rod may be sleeve-shaped, in which case the drive spring may be disposed at least partially in the plunger rod in order to reduce the size of the device. The release means may have an unlatching recess, for example, which is disposed on an axial level with the unlatching member when the release means is in an end position. Consequently, the retaining engagement of the unlatching member with the plunger rod can be released when the release means is in its end position or at least when the lock member has been released from the locking engagement with the release means. As a result of an oblique shape of the retaining shoulder or due to the spring force, the unlatching member, which may be mounted so as to be resilient and displaceable transversely to the longitudinal axis of the device, is able to move into the unlatching recess of the release means when the two are moved into axial alignment.

The retaining engagement of the unlatching member with the plunger rod may be released when the latching member is blocking the release element. In another preferred embodiment, the unlatching member additionally fulfils the function of the latching member. This being the case, at the same time as the retaining engagement is released, the release means is advantageously blocked in its end position by the unlatching member before being returned to its initial position.

In another embodiment, at least one of the lock member and unlatching member, and in some preferred embodiments both, is disposed on a sleeve formed by the housing or connected to the housing so that the sleeve is immobilised relative to the housing.

### BRIEF DESCRIPTION OF THE DRAWINGS

FIG. 1 is a diagram showing a longitudinal section of an automatic injector in accordance with the present invention with a release means and a contact element in an initial position,

FIG. 2 shows the automatic injector with the contact element in an intermediate position,

FIG. 3 shows the automatic injector with the contact element and the release element each in an end position,

FIG. 4 shows the automatic injector in an injecting position,

FIG. 5 shows the automatic injector in an injecting position and with the product dispensed,

FIG. 6 shows the automatic injector with an extracted needle covered by the contact element, and

FIG. 7 shows the automatic injector with a needle retracted into the housing covered by the contact element.

### DETAILED DESCRIPTION OF THE DRAWINGS

With regard to fastening, mounting, attaching or connecting components of the present invention to form an injection device or components thereof, unless specifically described otherwise, such are intended to encompass conventional fasteners such as threaded connectors, snap rings, detent arrangements, clamps, pins and the like. Components may also be connected by adhesives, glues, welding, ultrasonic welding, and friction fitting or deformation, if appropriate, and appropriate liquid and/or airtight seals or sealing devices may be used. Electronic features of the device, if any, may be commercially available electronic components, connectors and devices such as suitable wiring, connectors, printed circuit boards, microchips, sensors, inputs, outputs and the like. Unless specifically otherwise disclosed or taught, materials for making the present invention and/or components thereof may be selected from appropriate materials such as metal, metallic alloys, ceramics, plastics and the like, and appropriate manufacturing or production methods including casting, pressing, extruding, molding and machining may be used.

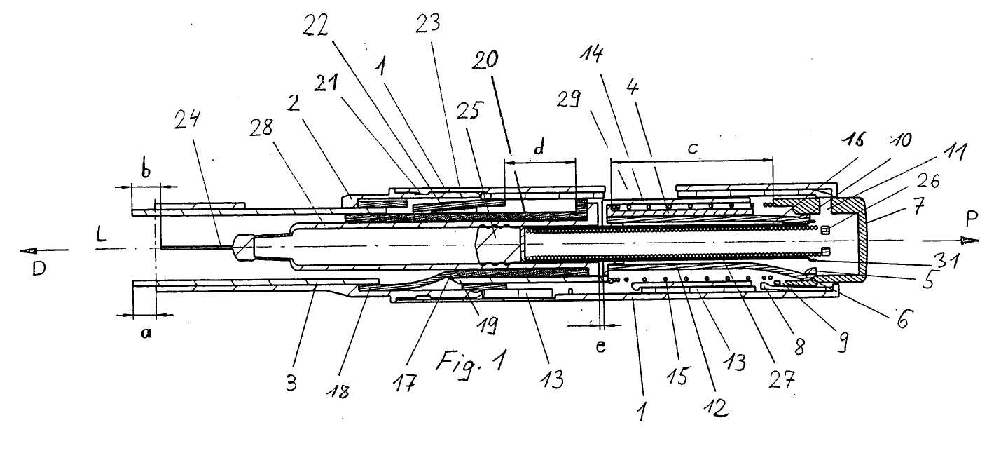

FIG. 1 illustrates an injection device proposed by the invention, comprising housing comprising a proximal housing part 1 and a distal housing part 2 connected to it. Since the embodiment illustrated is a disposable automatic injector, the two housing parts 1 and 2 are non-releasably connected to one another by means of a housing lock 21, i.e. the two housing parts 1 and 2 can not be detached from one another other than by breaking at least one of the two housing parts. Accommodated in the housing is an ampoule 28, which has a plunger 25 at its proximal end, which is longitudinally displaceable in the ampoule 28. A needle 24 is attached to the distal end of the ampoule. The ampoule 28 is accommodated in an ampoule holder 20, which can be displaced relative to the housing in the direction of the longitudinal axis L, in particular by the distance d. A release cam 23 is provided on the needle protector sleeve 3. The release cam 23 specifically has the shape of a fin or a wedge, which tapers in the distal direction and has an end face at its proximal end extending more or less perpendicular to the longitudinal axis L. The ampoule holder 20 has a cam or collar pointing radially outwards at its end pointing in the proximal direction P.

Disposed in the front housing part 1 is a contact element provided in the form of a needle protector sleeve 3, which is displaceable relative to the housing in the direction of the longitudinal axis L. The needle protector sleeve 3 projects out from the end of the distal housing part 2 pointing in the distal direction D by the distance a. The end of the needle protector sleeve 3 also projects out from the tip of the needle 24 by the distance b in the distal direction D. When the injection device is in the initial position, the distance a is shorter than the distance b.

Also disposed in the distal housing part 2 is a functional sleeve 18, which is axially displaceable relative to the housing part 2. The functional sleeve 18 has a lock arm 22 and a lock element 17. The lock element 17 is designed to be biased by the functional sleeve 18 via an arm. At its end pointing in the proximal direction P, the lock element 17 has a surface extending transversely to the longitudinal axis L, which co-operates with a complementary surface disposed on the ampoule holder 20 and forms a transmission mechanism. The lock element is advantageously biased via the arm of the functional sleeve 18 transversely to the longitudinal axis L so that the two transmission surfaces lie abutting with one another. The lock arm 22 disposed in the region of the release cam 23 is resiliently formed by the functional sleeve 18 and is biased so that it presses against the release cam 23.

Disposed at the proximal end of the proximal housing part 1 is a release means 7, which is displaceable relative to the housing part 1 along the longitudinal axis L. The release means 7 is sleeve-shaped and is closed at its proximal end, forming a knob. The release element 7 is pushed into a proximal position by the return spring 15, namely the initial position. The return spring 15 is supported at its end pointing in the distal direction D on a collar extending radially outwards from a sliding sleeve 4, which is displaceable along the longitudinal axis L in the proximal housing part 1. When the housing parts 1 and 2 are connected, the needle protector sleeve 3 and the sliding sleeve 4 abut with one another by their end faces. The return spring 15 pushes the sliding sleeve 4 and hence also the needle protector sleeve 3 in the distal direction D and holds the needle protector sleeve 3 in its initial position. Conversely, a movement of the needle protector sleeve 3 causes the sliding sleeve 4 to be driven in the proximal direction P. The return spring 15 is pushed back across the distance c.

The proximal housing part 1 also has a sleeve 12, which is essentially stationary relative to the proximal housing part 1. The sleeve 12 forms a lock member 6 on a resilient arm and is elastically displaceable transversely to the longitudinal axis L. At its end pointing in the proximal direction P, the lock member 6 has an oblique transmission surface, which co-operates with a transmission surface formed on the release means 7. The mutually abutting surfaces are able to slide on one another and thus form a transmitting mechanism capable of causing a longitudinal movement of the release means 7 to be converted into a movement of the lock member 6 directed transversely to the longitudinal axis L. In the state illustrated in FIG. 1, however, the lock member 6 is not able to move transversely to the longitudinal axis L because the lock member 6 is held in engagement with the release means 7 via the release member 5 disposed on the same level as the lock member 6 in the longitudinal direction L.

The sleeve 12 also has an unlatching member 10 provided on a resilient arm, which prevents a plunger rod 27 from moving in the distal direction D.

The plunger rod 27 is displaceable relative to the housing along the longitudinal axis L. The sleeve-shaped plunger rod is closed at its end pointing in the distal direction D or at least forms a circumferentially extending, e.g. annular, distal spring stop pointing radially inwards. The plunger rod co-operates with the plunger 25 by means of its end face pointing in the distal direction D. Disposed at the proximal end of the plunger rod is a shoulder 31 pointing radially outwards, in particular a circumferentially extending, annular shoulder 31. A drive spring 16 accommodated in the plunger rod is supported on a distal end of the plunger rod 27 and on a support stop. The support stop 26 may be provided by the housing 1 or, as illustrated in FIG. 1, by the sleeve 12. In the position illustrated, the drive spring 16 is in the biased state, pushed back in the longitudinal direction L. An oblique surface provided on the shoulder 31 of the plunger rod 27 is able to push the unlatching member 10 radially outwards due to the biasing force of the drive spring 16, but this is prevented by means of a radially abutting portion of the release means 7. When the plunger rod 27 is positioned as illustrated in FIG. 1, therefore, the drive spring 16 is not able to move it in the distal direction D.

The proximal housing part 1 surrounds a latch sleeve 13, which constitutes a latch member 8. The latch member 8 is disposed on a resilient arm of the latch sleeve 13 and can be elastically moved transversely to the longitudinal direction L. The latch member 8 has an oblique surface at its end directed towards the release means 7, which converts a longitudinal movement of the release means 7 into a movement of the latching member 8 directed transversely to the longitudinal axis L. The latch sleeve 13 is stationary relative to the housing part 1. The return spring 15 is surrounded by a sleeve-shaped display element 14. The display element 14 has a tubular portion, which is mounted so that it guides and can move the plunger rod 27 lengthways. Between a distal end of the display element 14 and the collar of the ampoule 28 pointing radially outwards is the distance e. The display element 14 may have a scale divided into portions or some other marking in the longitudinal direction, which can be viewed through a window 29 disposed in the housing part 1 and in the latch sleeve 13.

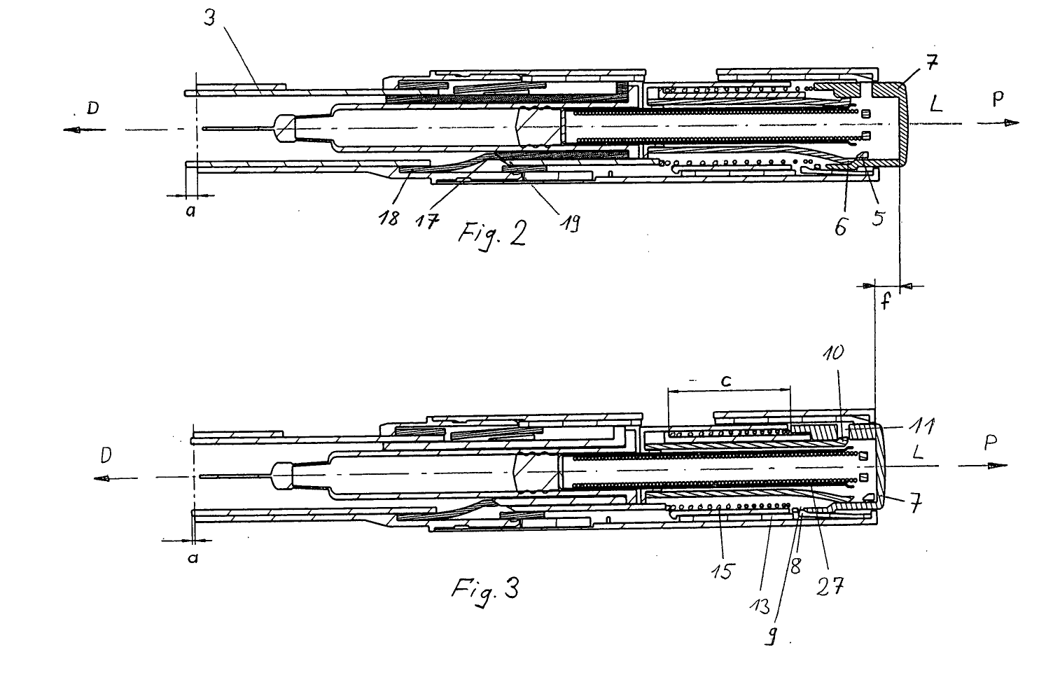

In the disposition illustrated in FIG. 1, the release means 7 can not be operated, namely the release means 7 can not be pushed in the distal direction D, because the release member 5 prevents the lock member 6 from effecting a movement radially inwards. In FIG. 2, the needle protector sleeve 3 has been pushed back in the proximal direction P, causing the release element 19 to release the lock element 17 of the functional sleeve 18 radially outwards. Due to the reduction in the distance a, the release member 5 moves in the proximal direction P because the needle protector sleeve 3 drives the sliding sleeve 4 with it. The return spring 15 is pushed back in the longitudinal direction L due to the reduction in distance a. The release member 5 has now released the lock member 6 at its side pointing radially inwards.

When the release means 7 is operated so that it moves by the distance f in the distal direction, an unlatching recess 11 disposed on the release means 7 is moved in the longitudinal direction L to the same height as the unlatching member 10, as illustrated in FIG. 3, so that the unlatching member 10 is able to latch in the unlatching recess 1 1. The shoulder 31 of the plunger rod 27 pointing radially outwards is now released from the unlatching member 10 at its end pointing in the distal direction D.

Also as a result of the release means 7 being operated, the lock member 6 has moved radially inwards by the co-operating transmission surfaces of the lock member 6 and release means 7, thus enabling the release means 7 to move into its end position illustrated in FIG. 3. As the release means 7 moves into its end position, the latch member 8 is pushed radially outwards due to its oblique surface, so that when the release means 7 is in the end position, the latch member 8 latches in the latching recess 9 formed by the release means 7. The release means 7 is no longer able to move back in the proximal direction P.

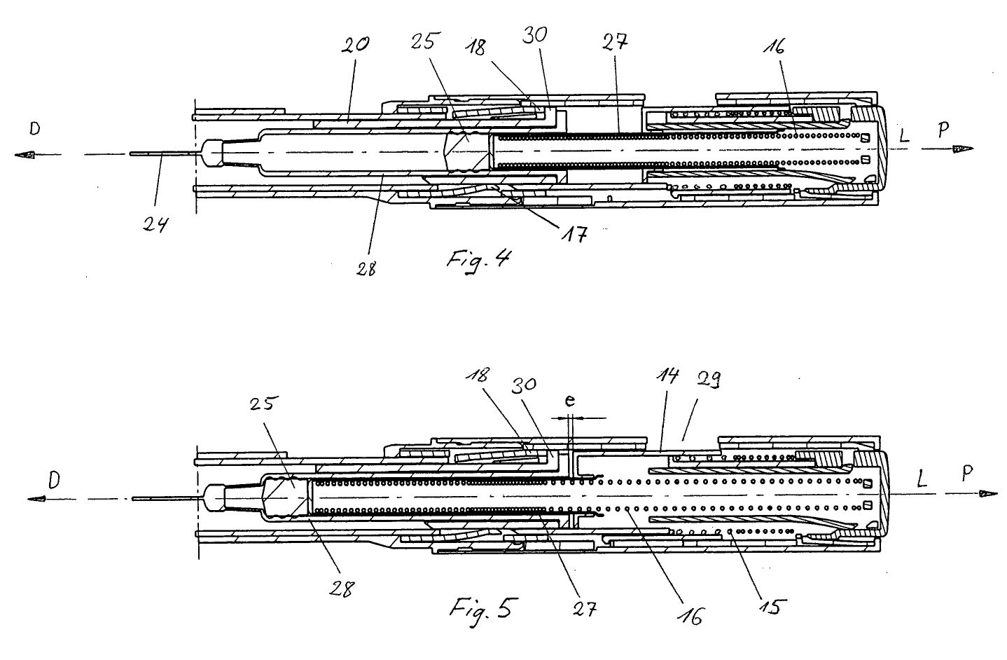

As illustrated in FIG. 4, the plunger rod 27 is pushed in the distal direction D, driven by the biased drive spring. The force exerted on the plunger 25 as a result acts on the preferably liquid product contained in the ampoule 28, but because of its viscosity and the fine needle passage of the needle 24, the force acting in the distal direction D is initially transmitted to the ampoule 28. Since the ampoule 28 is accommodated in the ampoule holder 20, which is disposed so as to be longitudinally displaceable in the distal housing part 2, the ampoule holder 20 incorporating the ampoule 28 and the needle is initially moved in the distal direction D, so that the needle 24 is able to pierce a body tissue. The ampoule holder 20 is pushed in the distal direction D by the distance d illustrated in FIG. 1 until the collar 30 abuts with the functional sleeve 18. Accordingly, the lock element 17 is pushed radially outwards by the surface of the ampoule holder 20 pointing in the distal direction D and now lies against the outer periphery of the ampoule holder 20 due to its inwardly acting biasing action.

As illustrated in FIG. 5, when the collar 30 is abutting with the functional sleeve 18, the plunger 25 is pushed in the distal direction D in the ampoule 28 by the drive spring 16 and the fluid contained in the ampoule 28 is dispensed. As the plunger rod 27 effects the dispensing movement, the display element 14 is driven with it via its tubular portion by the shoulder 31 of the plunger rod 27 directed radially outwards. A reading can be taken of the markings on the display element 14 through the window 29.

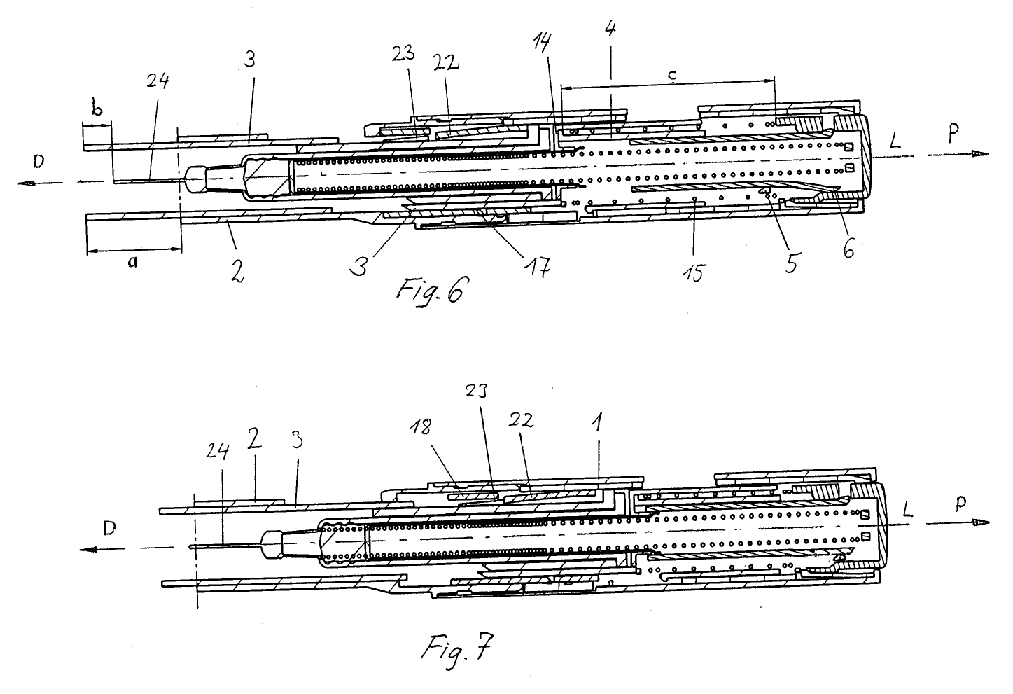

As illustrated in FIG. 6, as the needle 24 is extracted from the body tissue, the return spring 15 pushes the sliding sleeve 3 in the distal direction D, as a result of which the needle protector sleeve 3 is also moved in the distal direction D and pushes the lock element 17 radially outwards by means of its oblique portion pointing in the distal direction D. The needle protector sleeve 3 now covers the needle 24. In particular, the needle protector sleeve 3 projects beyond the tip of the needle 24 in the longitudinal direction L by the distance d. The distal end of the needle protector sleeve 3 is spaced at a distance a apart from the distal housing part 2. As the sliding sleeve 4 moves in the distal direction D, the release member 5 is also moved with it. The oblique transmission surface of the release member 5 pointing in the distal direction D is able to co-operate with the transmission surface of the lock member 6 and thus move the lock member 6 radially inwards so that the release member 5 is able to pass the lock member 6 in the longitudinal direction L. Also as the needle protector sleeve 3 moves in the distal direction D, the lock arm 22 is moved radially outwards by the transmission surface of the lock cam 23, as a result of which the lock cam 23 is able to pass the lock arm 22.

In order to save space when disposing of the injection device after use, the needle protector sleeve 3 can be moved back into the housing, as illustrated in FIG. 7, but without the needle 24 projecting out from the needle protector sleeve 3. As the needle protector sleeve 3 is moved back, the lock cam 23 moves so that its end pointing in the proximal direction P comes into abutment with the end face of the arm 22 pointing in the distal direction D and thus moves the functional sleeve 18 with it as the needle protector sleeve 3 continues to move. In order to prevent the needle protector sleeve 3 from being pushed back out of the housing due to the spring force of the drive spring 16, a locking lug is provided on the functional sleeve 18, which latches with the housing 1; 2 and axially blocks the functional sleeve 18 when the functional sleeve 18 or needle protector sleeve 3 has been moved back into the housing.

While multiple illustrative embodiments, including preferred embodiments, are disclosed, still other embodiments and/or aspects of the present invention will become apparent to those skilled in the art. As will be realized, the invention is capable of modifications in various obvious aspects, all without departing from the spirit and scope of the present invention. Accordingly, the drawings and detailed description are to be regarded as illustrative in nature and not restrictive, and all such modifications and variations are within the scope of the invention as determined by the appended claims when interpreted in accordance with the breadth to which they are fairly, legally, and equitably entitled.

1. An injection device comprising a contact element which is pressed onto a body tissue for an injection, a release for initiating product dispensing, which contact element and release are respectively displaceable along a longitudinal axis of the device between an initial position and an end position, wherein a movement of the release in at least one of the initial position of the contact element and the end position of the release can be prevented.

2. The injection device according to claim 1, further comprising a lock member for blocking the movement of the release along the longitudinal axis when the contact element is in the initial position.

3. The injection device according to claim 2, wherein the release comprises a cam mechanism and a movement of the release in the direction of the longitudinal axis causes a movement of the lock member generally transverse to the longitudinal axis.

4. An injection device for dispensing a product from a container which can be accommodated by the injection device, comprising:
a) a contact element which is pressed onto a body tissue for an injection,
b) release means for initiating product dispensing,
c) the contact element and release means respectively displaceable along a longitudinal axis of the automatic injector between an initial position and an end position, wherein
d) a movement of the release means in at least one of the initial position of the contact element and the end position of the release means can be blocked.

5. The automatic injector as claimed in claim 4, further comprising a lock member for blocking the movement of the release means along the longitudinal axis when the contact element is in the initial position.

6. The automatic injector as claimed in claim 5, wherein the lock member and the release means comprise elements of a cam mechanism and a movement of the release means in the direction of the longitudinal axis causes a movement of the lock member directed transversely to the longitudinal axis.

7. The automatic injector as claimed in claim 6, wherein the lock member and the release means each have a surface, and the surface of one is able to slide on the surface of the other.

8. The automatic injector as claimed in claim 5, wherein a movement of the contact element causes a movement of a release member relative to the lock member.

9. The automatic injector as claimed in claim 5, wherein, when the lock member is in a position in which it blocks a movement of the release means, the release member holds the lock member in a locking engagement with the release means.

10. The automatic injector as claimed in claim 9, wherein the release means is longitudinally displaceable relative to a housing and the lock member is coupled with the housing, and wherein the lock member can be moved against an elastic force by the release means out of the locking engagement with the release means.

11. The automatic injector as claimed in claim 5, further comprising a latch member for blocking a movement of the release means when the release means is in a position in which the lock member is not blocking a movement of the release means.

12. The automatic injector as claimed in claim 11, wherein the latch member is engageable with the release means in order to block the release means.

13. The automatic injector as claimed in claim 11, wherein the latch member is displaceable against an elastic force.

14. The automatic injector as claimed in claim 5, further comprising a plunger rod and an unlatching member, the unlatching member held in a retaining engagement with the plunger rod by the release means in order to block a dispensing movement of the plunger rod.

15. The automatic injector as claimed in claim 14, wherein the retaining engagement of the unlatching member with the plunger rod is released when the lock member has released itself from the locking engagement with the release means.

16. The automatic injector as claimed in claim 15, further comprising a latch member able to block a movement of the release means when the release means is in a position in which the lock member is not blocking a movement of the release means, wherein the retaining engagement of the unlatching member with the plunger rod is released when the latch member is blocking the release means.

17. The automatic injector as claimed in claim 16, wherein at least one of the lock member and unlatching member is one of disposed on a sleeve formed by the housing or connected to the housing so that the sleeve is immobilised with respect to the housing.

18. The automatic injector as claimed in claim 16, wherein the lock member and the unlatching member are one of disposed on a sleeve formed by the housing or connected to the housing so that the sleeve is immobilised with respect to the housing.
